(19) United States
(12) Patent Application Publication
(10) Pub. No.: US 2025/0122261 A1
(43) Pub. Date: Apr. 17, 2025

(54) INHIBITION OF NEURONAL ACTIVITY BY MEANS OF POTASSIUM ION CHANNELS

(71) Applicant: UNIVERSITA' DEGLI STUDI DI MILANO, MILANO (IT)

(72) Inventors: Anna MORONI, MILANO (IT); Lorenzo BROCCA, MILANO (IT); Andrea SAPONARO, MILANO (IT)

(21) Appl. No.: 18/692,437

(22) PCT Filed: Sep. 16, 2022
(86) PCT No.: PCT/IB2022/058761

§ 371 Date: Mar. 15, 2024

(30) Foreign Application Priority Data
Sep. 17, 2021 (IT) 102021000023987

(51) Int. Cl. C07K 14/705; A61K 38/00; C07K 14/005; C07K 14/195

(52) U.S. Cl. CPC C07K 14/705; C07K 14/005; C07K 14/195; A61K 38/00; C12N 2710/00022; C12N 2710/00033

(57) ABSTRACT

The present invention relates to a polypeptide which is a temperature-modulated ion channel, wherein said ion channel is a channel comprising four subunits which associate to form a homo- or hetero-tetramer, preferably is a potassium channel, wherein said polypeptide comprises at least one peptide comprising at least one portion of one of said subunits and a portion of the cytoplasmic C-terminal domain of the bacterial voltage-gated sodium channel BacNav, wherein said portion comprises at least one mutation in one of the amino acids in position 267, 271 or 274, wherein said numbering is read with reference to the voltage-gated sodium channel BacNav, SEQ ID NO: 5. The present invention further relates to the use of said polypeptide in the treatment of a condition modulated by potassium channel activity, selected from the group comprising: neuromyotonia, episodic ataxia type 1, hereditary deafness syndromes, benign familial neonatal seizures and periodic hypokalemic paralysis, neuropathic pain, diabetic neuropathic pain, trauma (thoracic surgery), glioblastoma, skin diseases, in particular originating from keratinocyte dysfunctions.

### BACKGROUND ART

Chronic pain (CP) is a medical condition affecting about 20% of adults in Europe, characterized by an abnormal duration of pain (>12 weeks) originally initiated by trauma or illness. When the source of the CP can be identified, conventional approaches based on the inhibition of ion channels, inhibition of inflammatory processes, and opioid analgesics are adopted to treat the specific pain symptoms. However, despite significant progress, CP remains extremely difficult to treat, with only two-thirds of patients reporting adequate pain relief.

The situation is even worse for neuropathic pain, a specific class of CP affecting 8% of the world population and the origin of which mainly depends on damage or a disorder of the peripheral or central nervous system, which leads the brain to interpret normally non-painful stimuli (allodynia) as pain or as an exaggerated response to the stimulus (hyperalgesia).

Neuropathic pain is particularly difficult to treat pharmacologically due to the large number of biological mechanisms involved: different cells, genes and proteins which work in synergy to transmit the signal, making it difficult to diagnose quickly and correctly the exact cause of the pain.

The inhibition of excitatory ion channels (e.g., sodium and calcium channels), although effective in rapid neuronal silencing for standard CP, has failed to treat neuropathic pain in a lasting manner due to the complex nature of the process, which hinders the development of an appropriate treatment protocol. On the other hand, central nervous system-targeted drugs (e.g., antidepressants and opioids) provide only partial pain relief and are nonspecific, also causing side effects such as addiction, nausea and respiratory depression, thus limiting the adoption thereof for prolonged treatments. New promising treatments currently being studied are based on optogenetic tools such as selective light-activated potassium (K⁺) channels (Alberio L et al. 2018 Nature Methods 15: 969-976). These are well-established tools for research purposes but still far from clinical application, due to the need for surgical optical fiber implantation.

Therefore, there is a strong need for alternative approaches to the treatment of CP and neuropathic pain, based on ion channels, which overcome the above limitations.

### DESCRIPTION

The present invention relates to an engineered ion channel which can be regulated by external stimuli which freely penetrate tissues and are safe for humans, avoiding the need for surgically implanted devices.

In a preferred embodiment, the ion channel is a potassium channel (K⁺) and the external stimuli which regulate it are temperature (T).

### DESCRIPTION OF THE DRAWINGS

FIGS. 1A-1C: Operating diagram of the T-sensitive channel according to the present invention. (FIG. 1A) structural model obtained with Alphafold2 of the engineered channel, formed by a portion of the ion channel for K⁺ KCV_{NTS} (AA 1-75) which forms the permeation pore for the ions through the membrane (in dark grey) and a portion of the bacterial channel BacNav Ale (AA 231-288, in light grey) which forms the temperature-sensitive domain and which comprises: part of the S6 helix and the CTD which includes the “neck” and the “coiled-coil”; (FIG. 1B) diagrammatic depiction of the closed channel inserted in the double lipid layer of the cell membrane; (FIG. 1C) open channel following an increase in temperature, with consequent passage of the K⁺ ions.

FIGS. 2A-2B: junction variants of the domains KCV_{NTS} and Ale and electrophysiological properties of the channels thus obtained. (FIG. 2A) comparison between the amino acid sequences of KCV_{NTS} (AA 1-82, SEQ ID NO: 4) and the temperature-sensitive channel TICK (SEQ ID NO: 8) or variant V thereof (SEQ ID NO: 9) constructed by modifying the length of KCV_{NTS} (in bold) and the S6 helix of Ale (underlined). (FIG. 2B) Patch clamp analysis at R.T. on HEK cells expressing the channel KCV_{NTS} and the TICK and variant V polypeptides.

FIGS. 3A-3D: Patch clamp analysis in HEK293 cells expressing the TICK channel. Measurements made (FIG. 3A) at 30° C.; (FIG. 3B) at 50° C.; (FIG. 3C) with the addition of 5 mM BaCl₂. The panel (FIG. 3D) shows the current-voltage curves derived from the three experimental conditions indicated.

FIGS. 4A-4B: Mutations inserted in the “coiled-coil” domain of the portion Ae1 to modify the T-dependency of the channel. (FIG. 4A) depiction of the 3D structure obtained with Alphafold2 of the temperature-sensitive peptide of Ae1, comprising “neck” and “coiled-coil”, the amino acids concerned in the mutations indicated on the right, numbered following the TICK sequence. (FIG. 4B) values of currents measured at the temperatures indicated in the wild-type channel (TICK) or in the channels with the indicated point mutations. Each point represents the amount of current measured in a cell.

FIGS. 5A-5B: Patch clamp analysis in HEK293 cells engineered with the channel TICK M112A (TICK1). Measurements made (FIG. 5A) at 37° C.; (FIG. 5B) at 39.5° C. The temperature increase was achieved by turning on an infrared lamp (IR lamp) about 20 cm away from the cells.

FIG. 6: Patch clamp analysis in HEK293 cells engineered with the channel TICK1, channel deactivation kinetics which was activated at 40° C. and then returned to 37° C.

FIGS. 7A-7C: Patch clamp analysis in HEK293 cells expressing (FIG. 7A) the channel TICK1; (FIG. 7B) the channel TICK1 S42T (comparative). Panel (FIG. 7C) shows the curves derived from the two experimental conditions indicated for the two samples.

### DETAILED DESCRIPTION OF THE INVENTION

The gating of ion channels is based on structural transitions between open and closed states. Potassium channels have a tetrameric structure, in which four subunits associate to form a complex arranged around a central ionic pore, where said four subunits modify the conformation thereof, leading to the activation/inactivation of the channel.

For the purpose of the present invention, it has been chosen to use the viral potassium channel encoded by the ATCV-1 KCV_{NTS} Chlorovirus, hereinafter referred to as KCV_{NTS}. This channel, which represents the pore model of K⁺ channels, has the advantage of being small and functionally very simple. Each subunit consists of two transmembrane domains, a sequence of selectivity and short N- and C-terminals. Despite the small size, KCV_{NTS} forms functional tetramers like other K⁺ channels and has many functional properties present in more complex K⁺ channels. These properties include potassium selectivity, a susceptibility to blockers, and the defined passage between open and closed state which however in this channel occurs randomly, i.e., it is not controlled by any cellular signal of a chemical (pH, calcium, ligand, . . . ) or physical nature (voltage, temperature, pressure, . . . ).

Said viral potassium channel encoded by the Chlorovirus ATCV-1 KCV_{NTS} has an amino acid sequence of 82 AA, SEQ ID NO: 4:

MLLLIIHLSILVIFTAIYKMLPGGMFSNTDPTWVDCLYFSASTHTTVGYG
DLTPKSPVAKLTATAHMLIVFAIVISGFTFPW.

The authors of the present invention surprisingly obtained a temperature-regulated potassium channel, with variations of a few degrees around the physiological temperature of mammals, using a portion of the potassium channel KCV_{NTS} and the cytoplasmic C-terminal domain (CTD) of the bacterial voltage-gated sodium channel BacNav Ae1, hereinafter referred to as Ae1, described in Arrigoni C et al. 2016 Cell 164: 922-936.

Said voltage-gated sodium channel BacNav Ae1 is encoded by an amino acid sequence of 288AA, SEQ ID NO: 5:

MSERQPDLVGHKVQHPEDETLRGRLAWFIDRPGTQYFIVGLILVNAITLG
LMTSPEVTAYLQPWLGWVNTFIIAAFVVEISLRIIADGPRFVRSGWNLFD
FSVVAISLVPDSGAFSVLRALRILKVLRLFSMVPRLRRIVEALLRAIPGI
AWIALLLLVIFYVFAVMGTKLFAQSFPEWFGTLGASMYTLFQVMTLESWS
MGIARPVIEAYPWAWIYFVSFILVSSFTVLNLFIGIIIESMQSAHWEAED
AKRIEQEQRAHDERLEMLQLIRDLSSKVDRLERRSGKR.

The CTD is formed by the “neck” and “coiled-coil”, encoding a temperature-sensitive peptide. As the temperature increases, the “neck” portion loses the conformation thereof, while the “coiled-coil” part, together with the S6 helix which precedes it in the Ae1 structure, keeps the structure more stable by constraining the “neck” and controlling the degree of unfolding thereof.

The authors of the present invention, having selected the portion of the channel KCV_{NTS} and the domain Ae1, conjugated the two by means of the final part of the S6 helix.

The portion of the channel KCV_{NTS} selected for the purpose of the present invention is encoded by SEQ ID NO: 6:

MLLLIIHLSILVIFTAIYKMLPGGMFSNTDPTWVDCLYFSASTHTTVGYG
DLTPKSPVAKLTATAHMLIVFAIVI

The domain Ae1 selected for the purpose of the present invention comprises the CTD and the last 13 amino acids of the S6 portion located upstream of the CTD. Said domain Ae1, which is the temperature-sensitive peptide, is encoded by SEQ ID NO: 7:

NLFIGIIIESMQSAHWEAEDAKRIEQEQRAHDERLEMLQLIRDLSSKVD
RLERRSGKR

Said conjugation has been shown to be an essential element for the functionality of the temperature-regulated potassium channel obtained by the conjugation itself. Such a junction was experimentally optimized by modifying the final portion of KCV_{NTS} and the initial portion of Ae1 to obtain a channel which, unlike the starting channel KCV_{NTS}, was closed at R.T. (room temperature), as evidenced by example 4 below.

Furthermore, three amino acid positions were identified on the “coiled-coil” domain on which point mutations were introduced. The mutations introduced surprisingly allowed the pore opening to occur by operating with elevations of a few degrees of temperature above the physiological temperature.

SEQ ID NO: 3 represents the domain Ae1 selected for the purpose of the present invention, in which the three amino acid positions on which one optionally intervenes with at least one mutation are indicated by X1, X2 and X3.

SEQ ID NO: 3
NLFIGIIIESMQSAHWEAEDAKRIEQEQRAHDERLEX1LQLX2RDX3
SSKVDRLERRSGKR

where X1, X2 and X3, which in the domain Ae1 have the following meaning: X1 is M, X2 is I, X3 is L, are optionally mutated independently of one another.

In an embodiment, at least one of the residues X1, X2, and X3, which in the domain Ae1 have the following meaning: X1 is M, X2 is I, X3 is L, is substituted with any other amino acid.

In an embodiment, said M in X1, said I in X2 and said L in X3 are optionally substituted with non-polar short-chain amino acids, preferably valine, alanine, or glycine, independently of one another.

In a further embodiment, said M in X1, said I in X2 and said L in X3 are optionally substituted with non-polar long-chain amino acids, such as leucine and isoleucine, or aromatic amino acids, such as phenylalanine, tyrosine, and tryptophan, independently of one another.

In a preferred embodiment, one of the amino acids between said M in X1, said I in X2 and said L in X3 is substituted with A.

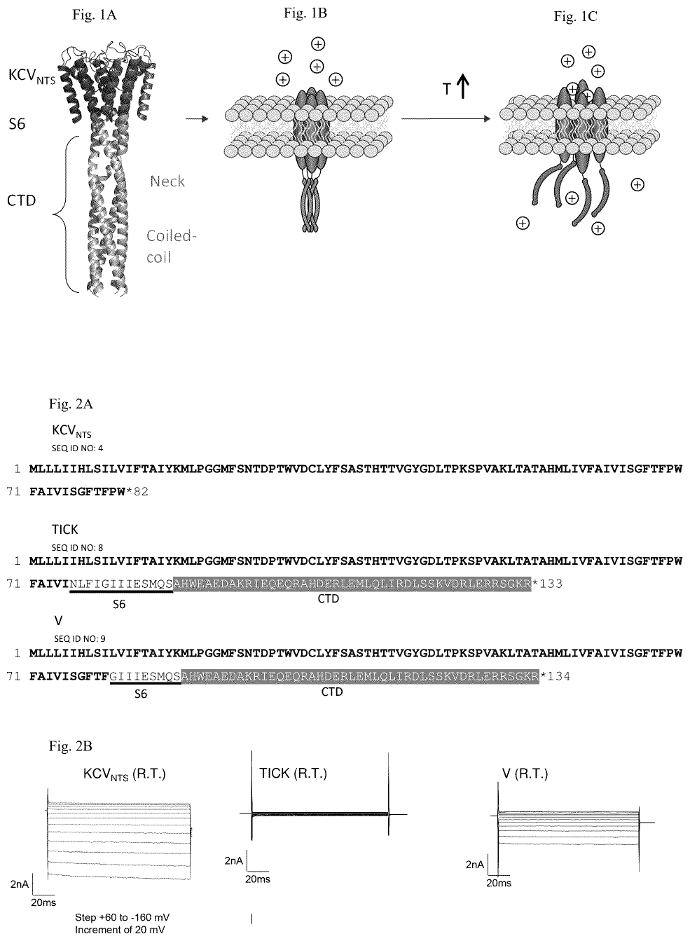

FIG. 1 diagrammatically shows an embodiment of an engineered channel according to the present invention. Panel A shows the 3D structure obtained with Alphafold2 of the engineered channel, shown in the tetrameric functional form thereof, where the following are indicated: the used portion of KCV_{NTS}, the S6 helix of Ae1, the CTD of Ae1 comprising the “neck” and the “coiled-coil”. In panels B, C, the tetramer channel is shown diagrammatically inserted in the double lipid layer of the cell membrane. In panel B the channel is closed, the four temperature-sensitive peptides (CTDs) form a “coiled-coil” which keeps the pore closed. As the temperature increases, the protein structure of the temperature-sensitive peptide (S6+CTD) tends to relax and rearrange leading to the opening of the pore, resulting in the passage of the potassium ions (FIG. 1C).

In a preferred form, said temperature-sensitive peptide, encoded by SEQ ID NO: 3 is directly connected to said portion of the channel KCV_{NTS} encoded by SEQ ID NO: 6.

The present invention first relates to a polypeptide which assembles to form an ion channel modulated by temperature variations, where said ion channel comprises four subunits which associate to form a homo-tetramer, where said polypeptide comprises at least said portion of KCV_{NTS}, SEQ ID NO: 6 and said domain Ae1, optionally with at least one mutation in one of positions 267, 271 or 274, where the numbering of said positions is read with reference to SEQ ID NO: 5, encoding BacNav Ae1, positions represented by X1, X2 and X3, respectively, in SEQ ID NO: 3.

In an embodiment, said ion channel is a homo-tetramer.

In an embodiment, said peptide comprising at least said portion of the channel KCV_{NTS} and said domain Ae1, comprising a portion of the S6 domain and all of the cytoplasmic C-terminal of the bacterial voltage-gated sodium channel BacNav has the sequence SEQ ID NO: 1

(I)
¹MLLLIIHLSILVIFTAIYKMLPGGMFSNTDPTWVDCLYFSASTHTTVGY
GDLTPKSPVAKLTATAHMLIVFAIVINLFIGIIIESMQSAHWEAEDAKRI
EQEQRAHDERLEX1LQLX2RDX3SSKVDRLERRSGKR¹³³,

where X1, X2 and X3, which in the domain Ae1 have the following meaning: X1 is M, X2 is I, X3 is L, are optionally mutated independently of one another.

In an embodiment, at least one of the residues X1, X2, and X3, which in the domain Ae1 have the following meaning: X1 is M, X2 is I, X3 is L, is substituted with any other amino acid.

In an embodiment, said M in X1, said I in X2 and said L in X3 are optionally substituted with non-polar short-chain amino acids, preferably valine, alanine or glycine, independently of one another, or said M in X1, said I in X2 and said L in X3 are optionally substituted with non-polar long-chain amino acids, such as leucine and isoleucine, or with aromatic amino acids, such as phenylalanine, tyrosine and tryptophan, independently of one another.

In an embodiment, one of the amino acids between said M in X1, said I in X2 and said L in X3 is substituted with A.

The authors of the present invention have shown that the potassium channel comprising the peptide of SEQ ID NO: 1 is finely controlled by temperature. In particular, said channel is activated when the temperature increases, with a half activation time (t 1%) of the order of seconds, and closes again, when returned to the initial temperature, within a few minutes. The same channel maintains responsiveness to barium, which inactivates it.

In an embodiment, when said sequence SEQ ID NO: 1 has said amino acid X1 which is a methionine (M112), said amino acid X2 116 which is I, said amino acid X3 which is L, the authors of the present invention have surprisingly demonstrated the responsiveness of said channel to temperature variations in the range 37° C., 50° C. Said channel is referred to, for the purpose of the present invention, as TICK and has SEQ ID NO: 8.

In a preferred form, when said sequence SEQ ID NO: 1 has said amino acid X1 which is an alanine (M112A), said amino acid X2 116 which is I, said amino acid X3 which is L, the authors of the present invention have surprisingly demonstrated the responsiveness of said channel to temperature variations in the range 37° C., 39.5° C., or in ranges applicable to mammals.

Said channel is referred to as TICK1 for the purpose of the present invention.

The numbering of positions 112, 116 and 119 is read with reference to SEQ ID NO: 3.

Therefore, the present invention relates to a polypeptide which forms a temperature-modulated tetramer potassium channel, where said polypeptide comprises at least one peptide having the amino acid sequence indicated by general formula (I), SEQ ID NO: 1

(I)
MLLLIIHLSILVIFTAIYKMLPGGMFSNTDPTWVDCLYFSASTHTTVGYG
DLTPKSPVAKLTATAHMLIVFAIVINLFIGIIIESMQSAHWEAEDAKRIE
QEQRAHDERLEX1LQLX2RDX3SSKVDRLERRSGKR

where X1, X2 and X3, which in the domain Ae1 have the following meaning: X1 is M, X2 is I, X3 is L, are optionally mutated independently of one another.

In an embodiment, at least one of the residues X1, X2, and X3, which in the domain Ae1 have the following meaning: X1 is M, X2 is I, X3 is L, is substituted with any other amino acid.

In an embodiment, said M in X1, said I in X2 and said L in X3 are optionally substituted with non-polar short-chain amino acids, preferably valine, alanine or glycine, independently of one another, or said M in X1, said I in X2 and said L in X3 are optionally substituted with non-polar long-chain amino acids, such as leucine and isoleucine, or with aromatic amino acids, such as phenylalanine, tyrosine and tryptophan, independently of one another.

In a preferred form, independently of one another, X1 is M or A, X2 is I or A, X3 is L or A.

In a preferred form, the polypeptide comprises the amino acid sequence SEQ ID NO: 2 and is the channel TICK1.

SEQ ID NO: 2:
MLLLIIHLSILVIFTAIYKMLPGGMFSNTDPTWVDCLYFSASTHTTVGYG
DLTPKSPVAKLTATAHMLIVFAIVINLFIGIIIESMQSAHWEAEDAKRIE
QEQRAHDERLEALQLIRDLSSKVDRLERRSGKR

In a further form, the polypeptide comprises the amino acid sequence of SEQ ID NO: 8 and is the channel TICK.

MLLLIIHLSILVIFTAIYKMLPGGMFSNTDPTWVDCLYFSASTHTTVGYG
DLTPKSPVAKLTATAHMLIVFAIVINLFIGIIIESMQSAHWEAEDAKRIE
QEQRAHDERLEMLQLIRDLSSKVDRLERRSGKR

The present invention further relates to a polynucleotide encoding at least one of the polypeptides according to the present invention.

The present invention further relates to a vector comprising said polynucleotide. In an embodiment, said polynucleotide is operatively conjugated to expression control sequences which allow the expression thereof in prokaryotic or eukaryotic host cells.

In a further embodiment, the use of the polypeptide according to the present invention for the treatment of a condition modulated by potassium channel activity is claimed herein. Said condition is selected from the group comprising: neuromyotonia, episodic ataxia type 1, hereditary deafness syndromes, benign familial neonatal seizures and periodic hypokalemic paralysis, neuropathic pain, diabetic neuropathic pain, trauma (thoracic surgery), glioblastoma, skin diseases, in particular originating from keratinocyte dysfunctions; in a preferred form it is chronic neuropathic pain. Baumbauer K M et al. 2015 in eLife Keratinocytes can modulate and directly initiate nociceptive responses, describe a direct role of keratinocytes in the modulation of nociceptive responses.

In an embodiment, the use of the temperature-sensitive potassium channel according to the present invention for the treatment of CP, in particular neuropathic pain, is claimed.

In a preferred form, said channel is administered by means of AAV (AdenoAssociated Virus).

Advantageously, the temperature-sensitive channel according to the present invention is expressed and active only in the specific neuronal cell, thus excluding undesirable interactions with other tissues. The engineered channel is produced directly by the host cell, which is engineered for example by means of AAV. AAV-based engineering techniques are now widely established, have a low risk of rejection or toxicity, and are therefore robust and safe techniques.

The method according to the present invention excludes the need for implants, where the channel is activated by the local temperature increase associated with inflammation or application of a hot patch and/or by exposure to an infrared lamp, or with ultrasound, making the invasiveness of this approach negligible.

Advantageously, the temperature-sensitive channel according to the present invention independently conducts the K+ current, with an extremely high conductance (about 100 pS) for each channel. This reduces the need for channel overexpression.

The T-sensitive channel according to the present invention is advantageously used in the use against neuropathic pain, since the closing times, of the order of tens of minutes, ensure that the activation remains for a sufficient time to obtain and maintain the analgesic effect induced by the outflow of potassium ions from the neuronal cells.

Experiments conducted in vivo in Zebrafish larvae have shown that the opening of the channel inhibits the movement of the larvae evoked by touch (touch response). Ex vivo experiments in slices of mouse brain have shown that the opening of the channel TICK1 caused by a rise in temperature inhibits firing in cortical neurons and that such an inhibition lasts for at least 30 minutes after the return to initial temperature conditions.

Furthermore, the opening of the potassium channels was found to significantly reduce neuropathic pain in a rat animal model.

The mechanism operated by the channel according to the present invention is physiological, allowing long neuronal inhibition times to be achieved without impacting cellular properties; for this reason, said channel is advantageously also used in treatments of chronic pain, for example in diabetic subjects, or is advantageously used as an adjunct in opioid treatments.

Unlike other thermoregulated potassium channels (TREK-1, TRAAK) which are already partially open at physiological temperature (37° C.) and which are further activated by both temperature and a range of different stimuli (polymodal), the channel according to the present invention is closed at physiological temperatures and responds exclusively to temperature variations, where such variations are compatible with mammalian physiology.

The following examples are for the sole purpose of exemplifying the invention, they are in no way to be understood as limiting the scope of protection of the present invention, the scope of which is defined by the claims.

### Example 1: Modulation of the TICK Channel in HEK 293 Cells

HEK293 cells expressing the TICK channel, SEQ ID NO: 8, where SEQ ID NO: 1 has X1=M, X2=I, X3=L, were analyzed by patch clamp, according to methods known to those skilled in the art.

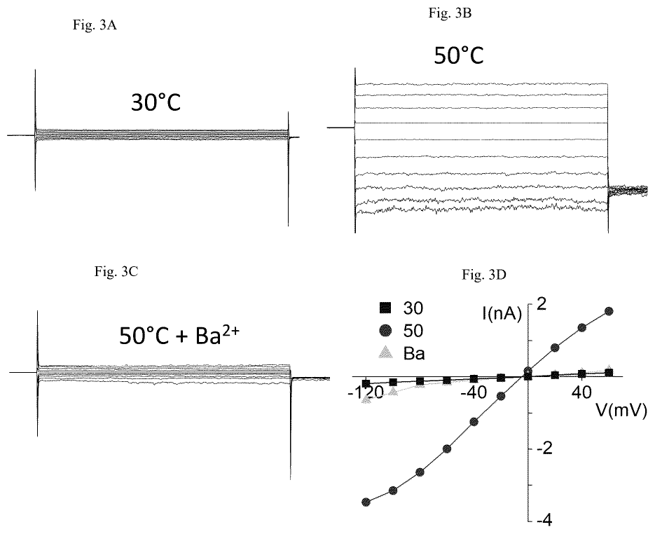

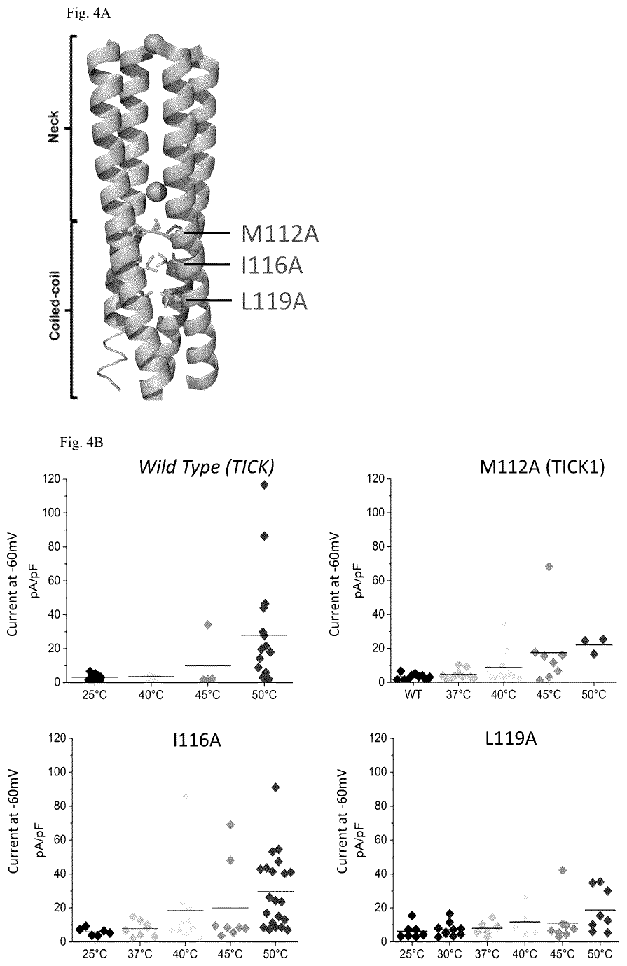

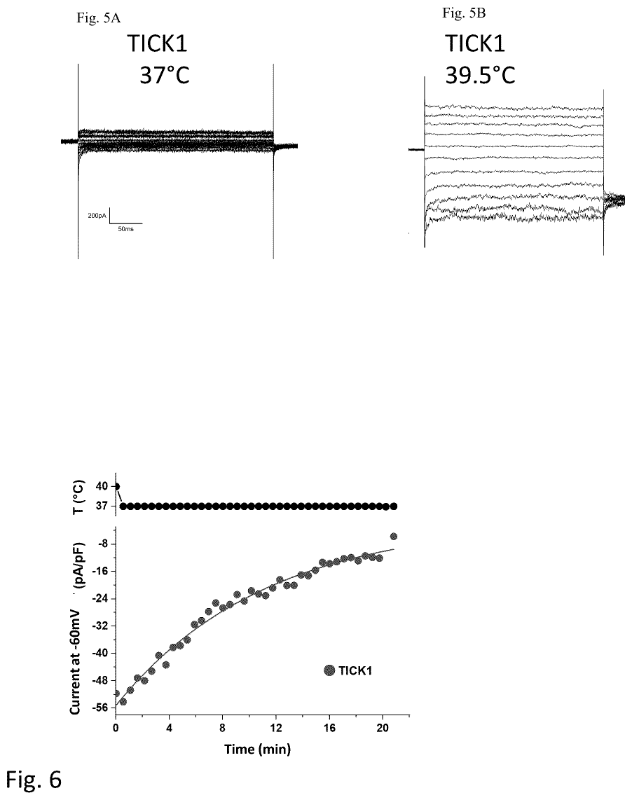

The results show that at 30° C., FIG. 3A, the channel is closed. By raising the temperature to 50° C., FIG. 3B, the passage of current is observed, indicative of the opening of the channel. The addition of Barium blocks the current, as expected for a K⁺ channel (FIG. 3C). In FIG. 3D, the current-voltage curves derived from the currents shown in panels A, B, C are shown.

### Example 2: Point Mutations on the Coiled-Coil Domain

Point mutations were introduced on the coiled-coil domain of the TICK channel in order to improve the temperature sensitivity thereof. Three mutants were obtained, in position 112, 116 and 119 with reference to SEQ ID NO: 3 (FIG. 4A). The three mutations independently introduced an alanine A substituting a methionine M, an alanine A substituting an isoleucine I, an alanine A substituting a Leucine L.

HEK 293 cells were transfected with said mutants, as well as with the mutation-free control (TICK). The currents measured at the different temperatures are shown in FIG. 4B. The replacements in position 116 were particularly advantageous and, even more advantageous, that in position 112 which allows observing an opening of the channel going from 37° C. to 40° C. This mutant, referred to as TICK1, SEQ ID NO: 2, was then selected for subsequent experiments, where the temperature variation range required for modulation is a range compatible with mammalian experiments. In the M112A graph in FIG. 4B, the first column shows the measurements performed on TICK, highlighting that the TICK1 mutant at the critical temperature of 37° C. behaves like the control, shown here at 40° C., and does not open. Conversely, by raising the temperature to 40° C., TICK1 opens.

The patch clamp experiment on HEK293 cells referred to in example 1 was repeated with HEK293 cells expressing the M112A mutant (TICK1), varying the temperature from 37° C. to 39.5° C. The results, shown in FIGS. 5A and 5B, respectively, surprisingly highlight the passage of current at a temperature of 39.5° C. (FIG. 5B), a passage not observed operating 2.5° C. below the opening T, or at 37° C. (FIG. 5A).

### Example 3: Channel Activation/Deactivation Kinetics

In HEK293 cells expressing the M112A mutant (TICK1) (FIG. 6), patch clamp measurements were performed using an infrared lamp as heat source which irradiated the preparation from a distance of 20 cm. The data in FIG. 6A show that the mutant channel opens following the marked temperature change. The opening of the channel occurs within seconds and reflects the temperature increase kinetics (not shown).

Furthermore, the deactivation kinetics are particularly slow, in the order of tens of minutes, as seen in FIG. 6. The graph shows the decrease over time of the current density of a cell exposed to 40° C., measured after bringing the temperature back to 37° C. A similar trend was measured in a set of cells (N=8), showing that in all the observed cells the closing of the potassium channels occurred with the timing shown in FIG. 5B.

### Example 4: KCV_{NTS} Conjugation with the Domain Ae1

In conjugating the portion of channel KCV_{NTS} with the domain Ae1, two different domains Ae1 were tested. A first domain, encoded by SEQ ID NO: 7, included 13 amino acids of the S6 helix, in addition to the CTD. A second domain, encoded by the sequence SEQ ID NO: 8, included 9 amino acids of the S6 helix in addition to the CTD and 5 additional C-terminal amino acids of KCV_{NTS} with respect to those present in the first domain. Said domains are compared in FIG. 2A.

Patch clamp experiments were performed on HEK293 cells as described in the preceding examples, where said HEK293 cells expressed KCV_{NTS} (SEQ ID NO: 4) not conjugated to a temperature-sensitive peptide, or a polypeptide where a portion of the channel KCV_{NTS} is conjugated to a domain Ae1 as in SEQ ID NO: 8 (TICK) or as in SEQ ID NO: 9 (variant V). The currents recorded at R.T. show that the construct SEQ ID NO: 8 is closed while the construct SEQ ID NO: 9 remains open, behaving like the control, or like the channel KCV_{NTS}. Surprisingly, the authors of the present invention have thus identified how to conjugate the channel portion to the temperature-sensitive peptide so as to have a polypeptide effectively capable of responding to temperature variations.

Variant V, SEQ ID NO: 9 (comparative, bold and underlining added in accordance with FIG. 2A)

MLLLIIHLSILVIFTAIYKMLPGGMFSNTDPTWVDCLYFSASTHTTVGY
GDLTPKSPVAKLTATAHMLIVFAIVISGFTFGIIIESMQSAHWEAEDAK
RIEQEQRAHDERLEMLQLIRDLSSKVDRLERRSGKR

### Example 5: TICK1 Mutation in Position S42 (Comparative)

The TICK1 mutant, according to the present invention, was mutated in position S42. The mutation involved replacing a Serine with a Threonine.

The TICK1-expressing HEK cells and the S42T mutant were analyzed by patch clamp, according to methods known to those skilled in the art.

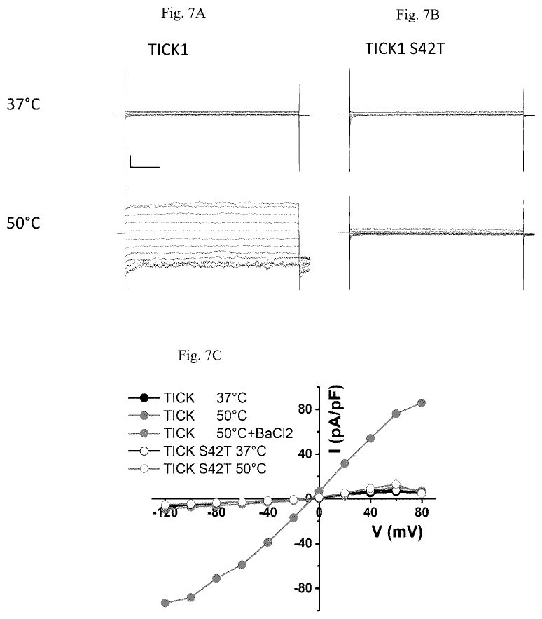

The results, shown in FIG. 7A for TICK1 and in FIG. 7B for TICK1 S42T, show that at 37° C., the channel is closed using both constructs. By raising the temperature to 50° C., the passage of current is observed, indicative of the opening of the channel only for TICK1, not for TICK1 S42T. FIG. 7C shows the current-voltage curves derived from the currents shown in panels A, B.

The data obtained here show how a single point mutation is capable of drastically impacting the functionality of the temperature-sensitive peptide, confirming the advantages of the mutations according to the present invention.

1-14. (canceled)

15. A polypeptide which assembles to form an ion channel modulated by temperature variations, wherein said ion channel comprises four subunits which associate to form a tetramer, wherein said polypeptide comprises at least one portion of a potassium channel and at least one portion of the voltage-gated sodium channel BacNav Ae1, optionally with at least one mutation in one of positions 267, 271 or 274, wherein the numbering of said positions is read with reference to SEQ ID NO: 5 encoding BacNav Ae1.

16. A polypeptide according to claim 15, wherein said potassium channel is the viral potassium channel encoded by the Chlorovirus ATCV-1 KCVNTS.

17. A polypeptide according to claim 15, wherein said portion of the channel KCVNTS is encoded by SEQ ID NO: 6.

18. A polypeptide according to claim 15, wherein said portion of Ae1 is encoded by SEQ ID NO: 3, where X1, X2 and X3 have the following meaning: X1 is M, X2 is I, X3 is L and they are optionally mutated independently of one another.

19. A polypeptide according to claim 18, wherein said M in X1, said I in X2 and said L in X3 are substituted with non-polar short-chain amino acids independently of one another.

20. A polypeptide according to claim 19, wherein said non-polar short-chain amino acids are selected among the group consisting of valine, alanine, or glycine.

21. A polypeptide according to claim 18, said M in X1, said I in X2 and said L in X3 are optionally substituted with non-polar long-chain amino acids, or aromatic amino acids, independently of one another.

22. A polypeptide according to claim 21, said non-polar long-chain amino acids being selected among the group consisting of leucine and isoleucine, said aromatic amino acids being selected among the group consisting of phenylalanine, tyrosine, and tryptophan.

23. A polypeptide according to claim 18, wherein X1 is M or A, X2 is I or A, X3 is L or A.

24. A polypeptide according to claim 18, wherein in at least one of said positions X1, X2, X3 the amino acid is substituted with respect to the amino acid found in SEQ ID NO: 5 encoding BacNav Ae l.

25. A polypeptide according to claim 23, wherein at least one of said positions X1, X2 or X3 is A.

26. A polypeptide according to claim 15, wherein said polypeptide comprises at least one peptide having the amino acid sequence indicated by general formula (I), SEQ ID NO: 1
(I)
MLLLIIHLSILVIFTAIYKMLPGGMFSNTDPTWVDCLYFSASTHTTVGYG
DLTPKSPVAKLTATAHMLIVFAIVINLFIGIIIESMQSAHWEAEDAKRIE
QEQRAHDERLEX1LQLX2RDX3SSKVDRLERRSGKR
optionally with at least one mutation in one of positions X1, X2 and X3, corresponding to positions 267, 271 or 274, wherein the numbering of said positions is read with reference to SEQ ID NO: 5 encoding BacNav Ae l.

27. A polypeptide according to claim 26, wherein in said peptide X1 is A, X2 is I, X3 is L, wherein said polypeptide is the T-sensitive channel TICK1.

28. A polynucleotide encoding at least one of the polypeptides according to claim 15.

29. A polynucleotide according to claim 28, which is operably conjugated to control sequences for the expression in prokaryotic or eukaryotic host cells.

30. A polypeptide according to claim 29, for use in the treatment of a condition modulated by potassium channel activity, selected from the group comprising: neuromyotonia, episodic ataxia type 1, hereditary deafness syndromes, benign familial neonatal seizures and periodic hypokalemic paralysis, neuropathic pain, diabetic neuropathic pain, trauma (thoracic surgery), glioblastoma, skin diseases, in particular originating from keratinocyte dysfunctions; in a preferred form it is chronic neuropathic pain.


SEQUENCE LISTING (9 sequences)

SEQ ID NO: 1 - protein, 133 aa, synthetic construct
  VARIANT 112
  VARIANT 116
  VARIANT 119
MLLLIIHLSILVIFTAIYKMLPGGMFSNTDPTWVDCLYFSASTHTTVGYGDLTPKSPVAKLTATAHMLIVFAIVINLFIGIIIESMQSAHWEAEDAKRIEQEQRAHDERLEXLQLXRDXSSKVDRLERRSGKR

SEQ ID NO: 2 - protein, 133 aa, synthetic construct
MLLLIIHLSILVIFTAIYKMLPGGMFSNTDPTWVDCLYFSASTHTTVGYGDLTPKSPVAKLTATAHMLIVFAIVINLFIGIIIESMQSAHWEAEDAKRIEQEQRAHDERLEALQLIRDLSSKVDRLERRSGKR

SEQ ID NO: 3 - protein, 58 aa, synthetic construct
  VARIANT 37
  VARIANT 41
  VARIANT 44
NLFIGIIIESMQSAHWEAEDAKRIEQEQRAHDERLEXLQLXRDXSSKVDRLERRSGKR

SEQ ID NO: 4 - protein, 82 aa, synthetic construct
MLLLIIHLSILVIFTAIYKMLPGGMFSNTDPTWVDCLYFSASTHTTVGYGDLTPKSPVAKLTATAHMLIVFAIVISGFTFPW

SEQ ID NO: 5 - protein, 288 aa, synthetic construct
MSERQPDLVGHKVQHPEDETLRGRLAWFIDRPGTQYFIVGLILVNAITLGLMTSPEVTAYLQPWLGWVNTFIIAAFVVEISLRIIADGPRFVRSGWNLFDFSVVAISLVPDSGAFSVLRALRILKVLRLFSMVPRLRRIVEALLRAIPGIAWIALLLLVIFYVFAVMGTKLFAQSFPEWFGTLGASMYTLFQVMTLESWSMGIARPVIEAYPWAWIYFVSFILVSSFTVLNLFIGIIIESMQSAHWEAEDAKRIEQEQRAHDERLEMLQLIRDLSSKVDRLERRSGKR

SEQ ID NO: 6 - protein, 75 aa, synthetic construct
MLLLIIHLSILVIFTAIYKMLPGGMFSNTDPTWVDCLYFSASTHTTVGYGDLTPKSPVAKLTATAHMLIVFAIVI

SEQ ID NO: 7 - protein, 58 aa, synthetic construct
NLFIGIIIESMQSAHWEAEDAKRIEQEQRAHDERLEMLQLIRDLSSKVDRLERRSGKR

SEQ ID NO: 8 - protein, 133 aa, synthetic construct
MLLLIIHLSILVIFTAIYKMLPGGMFSNTDPTWVDCLYFSASTHTTVGYGDLTPKSPVAKLTATAHMLIVFAIVINLFIGIIIESMQSAHWEAEDAKRIEQEQRAHDERLEMLQLIRDLSSKVDRLERRSGKR

SEQ ID NO: 9 - protein, 134 aa, synthetic construct
MLLLIIHLSILVIFTAIYKMLPGGMFSNTDPTWVDCLYFSASTHTTVGYGDLTPKSPVAKLTATAHMLIVFAIVISGFTFGIIIESMQSAHWEAEDAKRIEQEQRAHDERLEMLQLIRDLSSKVDRLERRSGKR